(19) United States
(12) Patent Application Publication
(10) Pub. No.: US 2019/0117272 A1
(43) Pub. Date: Apr. 25, 2019

(54) VARIABLE SCREW TOP CROSS CONNECTOR

(71) Applicant: Astura Medical, Carlbad, CA (US)

(72) Inventors: Keith Klausman, Carlsbad, CA (US); Thomas Purcell, Carlsbad, CA (US)

(73) Assignee: Astura Medical Inc., Carlbad, CA (US)

(21) Appl. No.: 16/167,442

(22) Filed: Oct. 22, 2018

Related U.S. Application Data
(60) Provisional application No. 62/575,469, filed on Oct. 22, 2017.

(51) Int. Cl. A61B 17/70

(52) U.S. Cl. CPC A61B 17/7049; A61B 17/7035; A61B 17/86; A61B 17/7011; A61B 17/7055

(57) ABSTRACT

A variable screw top cross connector for connecting spinal rods is provided that is designed to attach between spinal screws to add rigidity to a construct. The variable screw top cross connector includes multiple adjustment options (elongate (x axis)/pivot (x axis)/pivot (z axis)/medial-lateral pivot (y axis), and has a friction fit mechanism which ensures the components do not move from their desired location which allows for easier attachment of the components.

### CROSS-REFERENCE TO RELATED APPLICATIONS

This application claims the benefit of U.S. Provisional Application No. 62/575,469, filed Oct. 22, 2017, which is incorporated herein by reference.

### FIELD

The present invention relates generally to the field of surgery, and more specifically, to cross connector devices for connecting and maintaining a spaced apart relationship between pedicle screws with bone alignment rods.

### BACKGROUND

Certain spinal conditions, including a fracture of a vertebra and a herniated disc, indicate treatment by spinal immobilization. Spinal immobilization may include spinal surgery that unites two or more vertebrae to prevent them from moving independently of each other. The surgery uses a bone interface anchor, such as a pedicle screw, inserted into at least two spaced-apart vertebras, with a stabilization rod interconnecting the two or more anchors to stabilize the vertebras spanned by the anchors. To help stabilize lateral movement of the vertebrae, cross connectors may be connected between pedicle screws or rods.

Standard cross connectors do not have enough adjustment and/or lack the capability to lock all forms of adjustment once in position. Current devices also cannot maintain their position once adjusted and “flop around” during the installation process.

Thus, there is a need for a cross connector that will provide flexibility to a surgeon or other qualified professional when installing and adjusting this device to a patient. It is with this need in mind that the present invention was developed.

### SUMMARY OF THE INVENTION

Embodiments of the present invention are directed to a variable screw top cross connector for connecting spinal rods that is designed to attach between spinal screws to add rigidity to a construct. The variable screw top cross connector includes multiple adjustment options (elongate (x axis)/pivot (x axis)/pivot (z axis)/medial-lateral pivot (y axis), and has a friction fit mechanism which ensures the components do not move from their desired location which allows for easier attachment of the components. The screw top cross connectors may be used with screws, hooks, rod connectors or a variation of all, together to add rigidity to the construct.

In one embodiment, a variable screw top cross connector is presented which includes a left lateral clasp assembly having a lateral clasp body and a lateral hex nut, the left lateral clasp and hex nut being configured to fixedly engage a first spinal screw, a right lateral clasp assembly having a lateral clasp body and a lateral hex nut, the right lateral clasp and hex nut being configured to fixedly engage a second spinal screw, central locking connector assembly having a body, an engagement clevis extending through an opening in the body, and locking nut, a first curved connector shaft having a first end fixedly coupled to the central locking connector, and a second end rotatably coupled to the left lateral clasp assembly being configured to provide anterior/posterior movement between the first curved connector shaft and left lateral clasp assembly, and a second curved connector shaft a first end slidingly coupled to the clevis to the central locking connector configured to provide medial/lateral movement and converging/diverging rotational movement between the second curved connector shaft and first curved connector shaft, and a second end rotatably coupled to the right lateral clasp assembly being configured to provide anterior/posterior movement between the second curved connector shaft and right lateral clasp assembly.

Further embodiments, features, objects and advantages of the invention, as well as structure and operation of various embodiments of the invention, are disclosed in detail below with references to the accompanying drawings. There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims.

### BRIEF DESCRIPTION OF THE DRAWINGS

The present embodiments may be understood from the following detailed description when read in conjunction with the accompanying figures. It is emphasized that the various features of the figures are not necessarily to scale. On the contrary, the dimensions of the various features may be arbitrarily expanded or reduced for clarity.

FIG. 1 is a perspective view showing one embodiment of a variable screw top cross connector;

FIG. 2 is a cross-sectional view at A-A of FIG. 1;

FIG. 3 is a cross-sectional view at B-B of FIG. 2;

FIG. 4 is a human skeleton showing a variable screw top cross connector used with pedicle screws attached to the vertebrae;

FIG. 5 is a sectional view at C-C of FIG. 4, and

FIG. 6. is an exploded perspective view of FIG. 5.

### DETAILED DESCRIPTION

Embodiments of the invention will now be described with reference to the figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The variable screw top cross connector includes multiple adjustment options (elongate (x axis)/pivot (x axis)/pivot (z axis)/medial-lateral pivot (y axis), and has a friction fit mechanism which ensures the components do not move from their desired location which allows for easier attachment of the components. The screw top cross connectors are used to attach two screws, two hooks, two rod connectors or a variation of all, together to add rigidity to the construct.

FIGS. 1-3 illustrate one embodiment of a variable screw top cross connector 100 that includes a left lateral clasp assembly 102, a right lateral clasp assembly 104, lateral hex nuts 106, a central locking connector assembly 108 having a connector engagement clevis 110 and locking nut 112, a first curved connector shaft 114 having a “T” end 116, and a second curved connector shaft 118 having a “T” end 120.

The left and right lateral clasp assemblies 102, 104 include a lateral clasp 122 having a slot 124 sized for the first curved connector shaft 114 or second curved connector shaft 118 to extend through. The lateral clasps 122 are configured to wrap around and engage “T” ends 116 or 120 so that the lateral clasp 122 pivots or rotates 126 about the “T” ends 116 or 120 for adjustment. When the lateral hex nut 106 is tightened, the lateral clasps 122 are configured to lock the “T” ends 116 or 120 in the desired position. Locking the “T” ends 116, 120 also locks the first and second curved connector shafts 114, 118 at the screw head. A second end 114a of the first curved connector shaft 114 is further coupled to the central locking connector assembly 108. A second end 118a of the second curved connector shaft 118 slidingly extends through the clevis 110 allowing lateral movement 128 and rotational movement 130.

FIG. 2 shows a cross-sectional view at A-A of FIG. 1 showing more detail of the central locking connector assembly 108. The central locking connector assembly 108 includes a body 132 having a central opening 134 sized to receive a threaded portion 136 of the clevis 110. The locking nut 112 includes internal threads 138 configured to engage threaded portion 136. The clevis includes an opening 140 sized to accept the second curved connector shaft 118. Tightening the locking nut 112 pulls the clevis 110 upward and locks the second curved connector shaft 118 against the lower portion of the body 132.

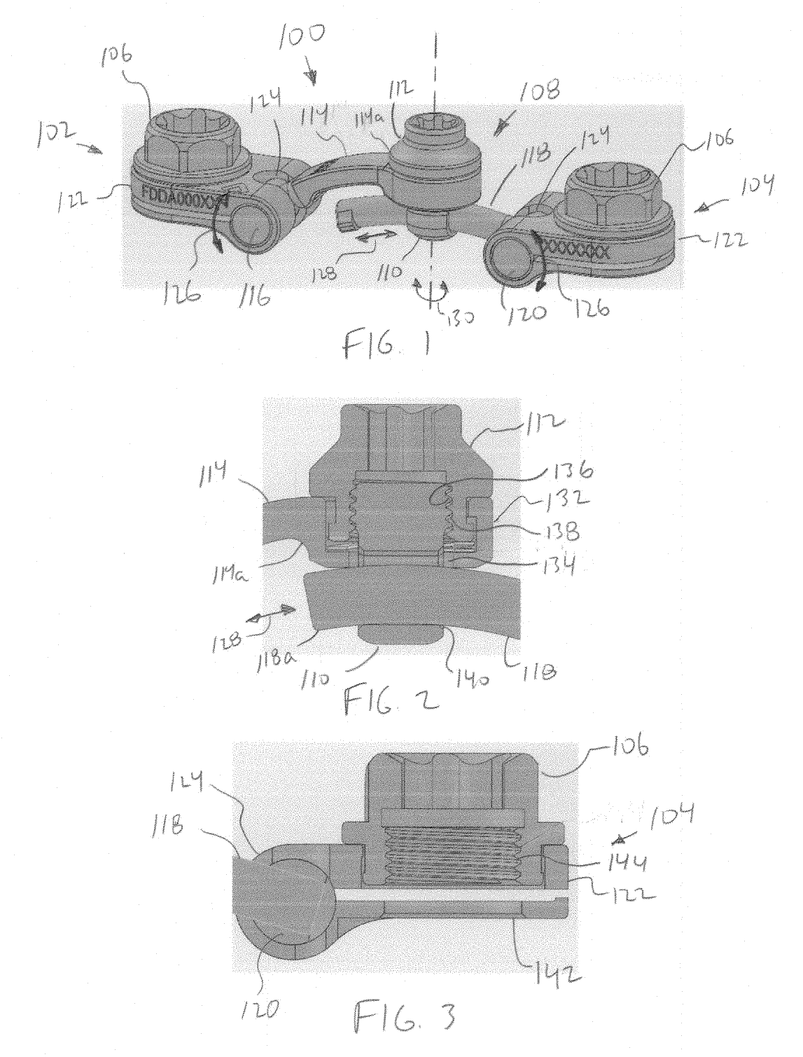

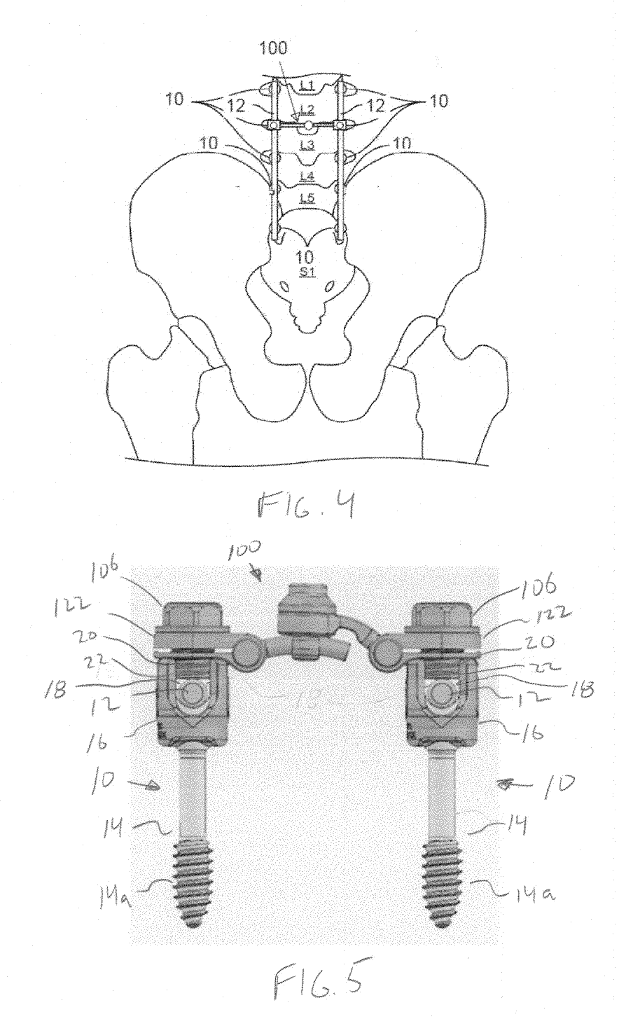

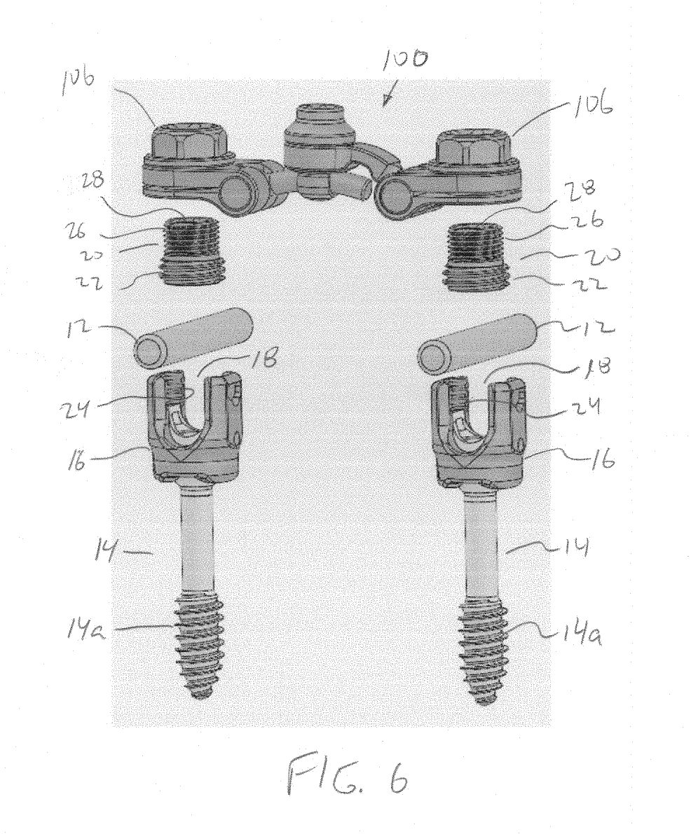

FIG. 3 shows a cross-sectional view at B-B of FIG. 1 showing more detail of the right lateral clasp assembly 104 engagement with the “T” end 120 of the second curved connector shaft 118. The lateral clasp 122 of the assembly includes an opening 142 sized to receive a threaded portion 22 of an extended height set screws 14 (shown in FIGS. 5 and 6). The lateral hex nut 106 includes internal threads 144 configured to engage threaded portion 22. Tightening the lateral hex nut 106 pulls the lateral clasp 122 closed against the “T” end 120 to lock it in the desired position. The lateral clasp assembly 102 is similar in design and includes the lateral clasp 122 engaging and locking “T” end 116.

FIG. 4 shows a portion of a human skeleton showing pedicle screws 10 attached to the vertebrae and sacrum. Spinal stabilization rods 12 connecting the pedicle screws 10 on either side of the vertebrae. The spinal screws 10 are configured to accommodate placement and securing of stabilization rods 12. The stabilization rods can be spinal connector rods or any other rods used in surgical procedures. The rods 12 can be secured at any angles and/or axial planes with regard to each other (e.g., rods can be converging, diverging, parallel, perpendicular to each other, etc.).

FIG. 5 is a sectional view at C-C of FIG. 4, and FIG. 6. is an exploded perspective view of FIG. 5. The spinal screws 10 include a shaft 14 with threaded portion 14a configured to engage the vertebrae coupled to a head portion 16 having a cavity 18 configured to accommodate placement of the spinal rods 12. The spinal rods 12 are secured within the cavities 18 by way of rod locking extended height set screws 20 having external threading 22. The upper portion of the cavities 18 include threading 24. The extended height set screws 20 are configured to be inserted into cavity openings 18 and setscrew threading 22 interacts with cavity threading 24. The extended height set screws 20 may also include external threading 26 configured to engage internal threading 144 within hex nuts 106 (see FIG. 3) The extended height set screws 20 also includes tool engagement cavities 28 in their top portions configured to accommodate insertion of a tool (not shown) that can be used by a surgeon or any other medical professional (or any other user) to “screw-in” setscrews 20 into the openings 18.

In use, once the rod 12 is placed inside the cavity 18, the user can insert the tool into the tool engagement cavity 28 to rotate the extended height set screws 20 in a downward direction. By rotating the extended height set screw 20 in the downward direction, the setscrew 20 pushes the rod 12 into the cavity 18 and further secures the rod 12 inside the cavity 18. As illustrated in figures, the setscrew 20 is configured to interact with the rod 12 and prevent rod 12 from falling out of the cavity 18. As can be understood by one skilled in the art, the rotation of the extended height set screws 20 can be in a clockwise or counterclockwise direction. Further, the cavity 18 and the extended height set screws 20 can be configured to accommodate any size spinal rod 12.

Once the spinal screws 10 are in place, the variable screw top cross connector 100 is positioned between the spinal screws 10 and the left and right lateral clasp assemblies 102, 104 are couple to the screws 10 by coupling the hex nuts 106 and extended height set screws 20. The lateral hex nuts 106 accommodate varying angled screws by way of the pivotable end clasps 122 and are both pivot able about the extended set screw 20. Further tightening the hex nuts 106 also tightens and locks the lateral claps 122 around “T” ends 116 and 120, locking them in place in the anterior/posterior direction.

The curved shaft 118 assembly slides through the clevis 110 and rotates within the clevis 110 allowing for width (medial/lateral) and off axis (converging/diverging) adjustability. The two sides are also capable of pivoting off of the central clevis to provide offset adjustability. By translating the rod 12 inside the clevis 110, a medical professional (e.g., surgeon) can adjust the distance between the spinal screws 10. When the centrally located nut 112 is tightened, the clevis 110 locks onto the second curved shaft 118, seizing variability. When the lateral hex nuts 106 are tightened, the lateral clasps 122 lock and also seize variability. The Variable Screw Top Cross Connector is composed of Ti6Al4V ELI titanium and Elgiloy (spring).

While embodiments and applications of this invention have been shown and described, it would be apparent to those skilled in the art that many more modifications than mentioned above are possible without departing from the inventive concepts herein. It is to be understood that the present disclosure is illustrative only and that changes, variations, substitutions, modifications and equivalents will be readily apparent to one skilled in the art and that such may be made without departing from the spirit of the invention as defined by the following claims.

The invention claimed is:

1. A variable screw top cross connector assembly comprising:
a left lateral clasp assembly fixedly coupled to a first spinal screw and rotateably coupled to a first end of a first connector shaft configured to provide anterior/posterior movement,
a right lateral clasp assembly fixedly coupled to a second spinal screw and rotateably coupled to a first end of a second connector shaft configured to provide anterior/posterior movement,
a central locking connector having a body fixedly coupled to the second end of the first connector; and
an engagement clevis having a connector shaft opening, the engagement clevis being rotatably coupled to the body to provide converging/diverging rotational movement and the shaft opening configured to slidingly receive the second end of the second connector shaft to provide medial/lateral movement.

2. The assembly of claim 1, wherein the left and right lateral clasp assemblies include slots sized for shafts of the first and second connector shafts to extend through, and the left and right lateral clasp assemblies are configured to wrap around and engage the “T” ends allowing for adjustment.

3. The assembly of claim 2, wherein lateral hex nuts are also configured to lock the rotation of the left and right lateral clasp assemblies and left “T” ends simultaneously in the desired anterior/posterior position.

4. The assembly of claim 1, further comprising a locking nut configured to engage the central locking connector and lock the engagement clevis in the desire converging/diverging position.

5. The assembly of claim 4, wherein locking the engagement clevis also locks the second end of the second connector shaft in the desire medial/lateral position.

6. The assembly of claim 1, wherein the first and second spinal screws include:
a shaft with threaded portion configured to engage a vertebrae;
a head portion couple to the shaft, the head portion having a cavity configured to accommodate placement of a spinal rod;
a rod locking set screw configured to secure the spinal rod within the cavity.

7. The assembly of claim 6, wherein rod locking set screw includes an extended height portion configured to engage the hex nuts and clamp the left and right lateral clasp assemblies to the screw head portions.

8. A variable screw top cross connector assembly comprising:
a left lateral clasp assembly having a lateral clasp body configured to fixedly engage a first spinal screw;
a right lateral clasp assembly having a lateral clasp body configured to fixedly engage a second spinal screw;
central locking connector assembly having a body, an engagement clevis extending through an opening in the body, and locking nut;
a first connector shaft having a first end fixedly coupled to the central locking connector, and a second end rotatably coupled to the left lateral clasp assembly being configured to provide anterior/posterior movement between the first connector shaft and left lateral clasp assembly; and
a second connector shaft a first end slidingly coupled to the clevis to the central locking connector configured to provide medial/lateral movement and converging/diverging rotational movement between the second connector shaft and first connector shaft, and a second end rotatably coupled to the right lateral clasp assembly being configured to provide anterior/posterior movement between the second connector shaft and right lateral clasp assembly.

9. The assembly of claim 8, wherein the left and right lateral clasp assemblies include slots sized for shafts of the first and second connector shafts to extend through, and the left and right lateral clasp assemblies are configured to wrap around and engage the “T” ends allowing for adjustment.

10. The assembly of claim 9, further comprising lateral hex nuts configured to lock the rotation of the left and right lateral clasp and the rotation of the “T” ends.

11. The assembly of claim 9, further comprising a locking nut configured to engage the central locking connector and lock the engagement clevis in the desire converging/diverging position.

12. The assembly of claim 11, wherein locking the engagement clevis also locks the second end of the second connector shaft in the desire medial/lateral position.

13. The assembly of claim 8, wherein the first and second spinal screws include:
a shaft with threaded portion configured to engage a vertebrae;
a head portion couple to the shaft, the head portion having a cavity configured to accommodate placement of a spinal rod;
a rod locking set screw configured to secure the spinal rod within the cavity.

14. The assembly of claim 13, wherein rod locking set screw includes an extended height portion configured to engage the hex nuts and clamp the left and right lateral clasp assemblies to the screw head portions.

15. A method of using a variable screw top cross connector assembly comprising:
inserting first and second spinal screws into the vertebrae in a desired location;
coupling spinal rods to the first and second spinal screws;
coupling a variable screw top cross connector assembly to the first and second spinal screws, the cross connector assembly including:
a left lateral clasp assembly fixedly coupled to the first spinal screw and rotateably coupled to a first end of a first connector shaft configured to provide anterior/posterior movement;
a right lateral clasp assembly fixedly coupled to a second spinal screw and rotateably coupled to a first end of a second connector shaft configured to provide anterior/posterior movement;
central locking connector assembly having a body rotateably coupled to a first end of a first connector shaft,
an engagement clevis rotateably coupled to the body converging/diverging rotational movement, the central locking connector being slidingly coupled to a second end of the second connector shaft provide medial/lateral movement;
adjusting the variable screw top cross connector to a desired configuration using anterior/posterior movement, converging/diverging rotational movement and medial/lateral movement; and
securing the variable screw top cross connector in the desired position.

16. The method of claim 17, wherein the left and right lateral clasp assemblies include slots sized for shafts of the first and second connector shafts to extend through, and the left and right lateral clasp assemblies are configured to wrap around and engage the “T” ends allowing for adjustment.

17. The method of claim 16, wherein lateral hex nuts are also configured to lock the first and second spine screws in the right and left “T” ends in the desired anterior/posterior position.

18. The method of claim 16, further comprising a locking nut configured to engage the central locking connector and lock the engagement clevis in the desire converging/diverging position.

19. The method of claim 18, wherein locking the engagement clevis also locks the second end of the second connector shaft in the desire medial/lateral position.

20. The method of claim 16, wherein the first and second spinal screws include:
a shaft with threaded portion configured to engage a vertebrae;
a head portion couple to the shaft, the head portion having a cavity configured to accommodate placement of a spinal rod;
a rod locking set screw configured to secure the spinal rod within the cavity.
